(19) United States
(12) Patent Application Publication
(10) Pub. No.: US 2007/0269402 A1
(43) Pub. Date: Nov. 22, 2007

(54) Aerosol total release container and method of use for odor elimination

(76) Inventor: Louis B. Johnson, Troy, AL (US)

Correspondence Address: CLARK & BRODY, 1090 VERMONT AVENUE, NW, SUITE 250, WASHINGTON, DC, 20005, US

(21) Appl. No.: 11/785,161

(22) Filed: Apr. 16, 2007

Related U.S. Application Data
(60) Provisional application No. 60/801,431, filed on May 19, 2006.

(51) Int. Cl. A61L 9/01; A61L 9/03

(52) U.S. Cl. 424/76.2

(57) ABSTRACT

An odor eliminating device comprises a total release aerosol container that contains an odor elimination compound in the container. The container is placed in an enclosed space that has an odor problem. The container is triggered to release tie odor elimination compound into the enclosed space to cure the odor problem.

This application claims priority under 35 USC 119(e) based on provisional patent application No. 60/801,431 filed May 19, 2006, which is incorporated herein by reference.

### TECHNICAL FIELD

The present invention relates to an aerosol total release container for odor elimination and its method of use.

### BACKGROUND ART

In the prior art, various techniques have been proposed to address the problems of odors, for example, cooking odors, pet odors, smoke odors, odors inherent to a material, waste odors, and the like. Some techniques use masking components so that the odor, while still present, cannot be smelled and is therefore not objectionable. Other techniques use adsorbents or absorbents that capture the offending compound so that it is not present to cause the odor problem. While these techniques can be effective, they do not offer the required odor elimination in certain situations. For example, in hunting, it is necessary to minimize the presence of any and all odors since the keenness of an animal's sense of smell will detect these odors and avoid the odor-emanating source. As an example, a hunting blind may be put in use so that a hunter can wait for the approach of game. Even though the hunter may use odor-eliminating techniques such as wearing the appropriate clothing or treating the clothing, the hunting blind may still have odors that would give away the hunter's location.

Other instances where odor is a problem is in hotel rooms, kitchens, bathrooms, bedroom, rooms occupied by pets, or virtually any room wherein an occupant, human or animal, can generate odors. It is not easy to rid these rooms of odors without an entire and thorough cleaning of all of the room components, e.g., walls, floors, fixtures, mattresses, chairs and sofa cushions, pillows, carpeting, wallpaper, and the like.

In view of the continuing problem with odors, a need exists to provide improved odor elimination techniques, including commercial residential structures, vehicles, portable enclosures where an enclosed space has an odor problem that must be solved quickly.

The present invention responds to this need by providing an odor elimination technique that is easily used in a confined space and provides good odor elimination.

### SUMMARY OF THE INVENTION

One object of the present invention is a total release aerosol container designed to eliminate or reduce odors in spaces such as a room, vehicles, structures, hunting enclosures, and the like.

Another object of the invention is a method of eliminating odors in a confined space by discharge of an odor eliminating compound using a total release aerosol container.

Other objects and advantages will become apparent as a description thereof proceeds.

In satisfaction of the foregoing objects of the invention, the present invention relates to a method of eliminating odors in an enclosed space. As part of this method, an active odor eliminating compound is charged into a total release aerosol container. The total release aerosol container is positioned in an enclosed space and triggered to discharge substantially all of the active odor eliminating compound to reduce odors in the enclosed space. The enclosed space can be any type, with examples including a hunting enclosure such as a hunting blind, a tent, a hotel room, and a bathroom.

The odor eliminating compound comprises an active odor eliminator component such as cyclodextrine, metazine, sodium or potassium carbonate, enzymes, silver or silver compounds, quaternary ammonium compounds, ozone, a synthetic or natural zeolite, or combinations thereof.

The invention also comprising the total release aerosol container that is pressurized and adapted to release substantially all of the active odor eliminating compound charged to the container compound to atmosphere upon triggering.

The inventive method and device also provide for a rapid release of the odor eliminating compound so that odor elimination occurs in the designated space in a fairly short period of time. This is accomplished by ensuring that the aerosol container discharges the odor eliminating compound in a continuous and rapid manner so that the odor elimination takes place quickly.

### DESCRIPTION OF THE PREFERRED EMBODIMENTS

The present invention offers significant advantages in the area of odor elimination for confined spaces. While prior art systems rely on targeted methods for odor elimination such as spraying a liquid into the air, e.g., Lysol® or the like, or metering defined amounts of a liquid over time, the present invention provides a much more expansive odor elimination capability. This expansive capability can be beneficial when confined spaces that suffer from chronic odor need a treatment or odor must be eliminated such as in a hunting environment. Another feature of the invention is the ability to eliminate odors in a rapid fashion.

These advantages are attained through the use of a total release aerosol device that contains an active odor eliminating compound as a component thereof.

Total release aerosol containers by themselves are known. An example of one is Doktor Doom Total Release Fogger, which contains the active ingredient pyrethrum for treating insects and can be purchased at different places, e.g., from www.homeharvest.com.

The active ingredient for the total release aerosol container of the invention is an active odor eliminating compound. This compound can be any one of a number of odor eliminators such as cyclodextrine, metazine, sodium or potassium carbonate, enzymes, silver or silver compounds, quaternary ammonium compounds, ozone, a synthetic or natural zeolite or a combination thereof. These compounds can be generally classified as adsorbents like the zeolites or absorbents like potassium or sodium carbonates, or metazine or capturing agents as cyclodextrines and the like. Cyclodextrines are believed to be especially beneficial for odor elimination due to their capturing capability.

The active odor eliminating compounds would be charged into the total release aerosol container in the same manner as other compounds are normally done. Since this charging process is well known, there is no need for a further discussion for understanding of the invention. In addition, other components as would normally be included in total release aerosol containers could also be included. If the aerosol container is intended for a hunting environment, any other components to be used with the active odor eliminator should be odorless as well so as not to defeat the purpose of the discharge of the active odor elimination. When using the aerosol container in an enclosed space where it is not critical to maintain it odor free, the active odor eliminator could be combined with other compounds, e.g., a perfume or other scent that would impart a pleasing scent to the enclosed space being treated. Since these types of scented compounds are well known, a further description is not necessary for understanding of this aspect of the invention.

In use, an area needing odor control would be first identified. This area can be virtually any area that could be treated with a total release aerosol container. Examples include hotel or motel rooms, bathrooms, hunting structures such as blinds, tents, enclosed tree stands and the like. The use of the total release aerosol containers in hunting structures is particularly advantageous since the elimination of odors when hunting is a prime goal for success.

Once the area to be treated is identified, the total release aerosol container having the active odor elimination compounds and the necessary pressurized materials for expulsion is placed in the desired location. The space is vacated and the container is triggered to expel the active odor elimination compound into the space for odor elimination. After a period of time when the compound has dispersed for odor elimination, the odor-free space can be occupied.

When it is important to have a rapid treatment, the aerosol container can be designed so that the total discharge of container material occurs in a brief period of time. For example, an aerosol container will take a minute or so to discharge, and the odor elimination effect is obtained in several minutes afterward.

As such an invention has been disclosed in terms of preferred embodiments thereof, which fulfills each and every one of the objects of the invention as set forth above, and provides an improved odor elimination device and method of use.

Of course, various changes, modifications and alterations from the teachings of the present invention may be contemplated by those skilled in the art without departing from the intended spirit and scope thereof. It is intended that the present invention only be limited by the terms of the appended claims.

I claim:

1. In a method of eliminating odors in a enclosed space, the improvement comprising providing an active odor eliminating compound and charging a total release aerosol container with the active odor eliminating compound, and discharging substantially all of the active odor eliminating compound from the total release aerosol container in an enclosed space.

2. The method of claim 1, wherein the enclosed space is a one of a hunting blind, hunting tree stand, a hotel room, a bathroom, a kitchen, a vehicle, and a bedroom.

3. The method of claim 1, wherein the odor eliminating compound includes one of cyclodextrine, metazine, enzymes, silver or silver compounds, quaternary ammonium compounds, ozone, sodium or potassium carbonate, a synthetic or natural zeolite or a combination thereof as the active odor eliminator.

4. The method of claim 2, wherein the active odor eliminating compound includes one of cyclodextrine, enzymes, silver or silver compounds, quaternary ammonium compounds, ozone, metazine, sodium or potassium carbonate, a synthetic or natural zeolite or a combination thereof as the active odor eliminator.

5. The method of claim 1, wherein the odor eliminator is discharged from the container in a continuous and rapid manner.

6. The method of claim 1, wherein the odor elimination is cyclodextrine.

7. An odor eliminating device comprising:
a pressurized container adapted to release substantially of its contents in an enclosed space upon triggering; and
an active odor eliminating compound charged to the container as part of the contents for release into the enclosed area for odor elimination or reduction.

8. The odor eliminating device of claim 7, wherein the odor eliminating compound includes one of cyclodextrine, enzymes, silver or silver compounds, quaternary ammonium compounds, ozone, metazine, sodium or potassium carbonate, a synthetic or natural zeolite or a combination thereof as the active odor eliminator.

9. The odor eliminating device of claim 7, wherein the aerosol container is adapted to discharge the odor eliminating compound in a continuous and rapid manner.

10. The odor eliminating device of claim 7, wherein the odor eliminating compound is cyclodextrine.
